(19) United States
(12) Patent Application Publication
(10) Pub. No.: US 2010/0160932 A1
(43) Pub. Date: Jun. 24, 2010

(54) MARKER ATTACHING DEVICE FOR ATTACHING A MARKER DEVICE

(76) Inventors: Michael Gschwandtner, Munchen (DE); Troels Frimor, Munchen (DE); Antonia Henning, Neukeferloh (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/640,517

(22) Filed: Dec. 17, 2009

Related U.S. Application Data
(60) Provisional application No. 61/155,967, filed on Feb. 27, 2009.

(30) Foreign Application Priority Data
Dec. 19, 2008 (EP) 08172335.5

(51) Int. Cl. A61B 17/00; B23Q 3/00

(52) U.S. Cl. 606/139; 29/283.5

(57) ABSTRACT

The present application relates to a marker attaching device for attaching a marker device to an object, comprising a part for fastening a marker device and comprising an attaching part for attaching the marker attaching device, wherein the attaching part can be deformed in order to enter into a mechanical connection with the object.

### RELATED APPLICATION DATA

This application claims the priority of U.S. Provisional Application No. 61/155,967, filed on Feb. 27, 2009, which is hereby incorporated in its entirety by reference.

### FIELD OF THE INVENTION

The present invention relates to a marker attaching device for attaching a marker device to an object, in particular to a medical instrument and/or an anatomical body structure and/or an implant, comprising a part for fastening a marker device and comprising an attaching part for attaching the marker attaching device to the object.

### BACKGROUND OF THE INVENTION

Modern operation procedures in the field of image-guided surgery (IGS) are often performed with the aid of a medical navigation and/or tracking system. In most cases, navigation is based on comparing at least one multi-dimensional model of at least parts of the patient's body with data of at least parts of the patient's body which is captured during the medical incision, wherein the reference dataset—which can include data concerning at least the geometry of at least one part of the patient's body and on which the model is based—is recorded before the operation with the aid of a medical imaging method. A reference dataset comprising geometric information concerning an implant and/or a medical instrument can also be provided. A marker device which is attached to an anatomical body structure of the patient and/or a medical instrument and/or an implant (all three are referred to in the following as the object; the terms “anatomical body structure”, “medical instrument” and/or “implant” are intended to be exchangeable within the framework of this text with respect to combining them with the marker attaching device in accordance with the invention) during the operation then serves for comparing the current position of the object with the reference dataset of the patient's body and/or the reference dataset of the instrument and/or implant, thus enabling navigation during the operation.

U.S. Pat. No. 6,190,395 B1 shows an attaching part for a surgical navigation system comprising an annular attaching part for a marker device. In accordance with claim 3 of this document, the annular clamping band is produced from a stainless steel foil having a thickness of about 5 to 50 mm. This embodiment does not allow the attaching part to be universally adaptable to an instrument surface or the surface of an anatomical structure.

U.S. Pat. No. 6,434,507 B1 shows an adaptor for an instrument head, using which the instrument head can be attached to a handle. A marker device can then be attached to the far end of the instrument from the adaptor. In this case, the difference with respect to the subject in accordance with the invention is on the one hand that a constituent of the instrument which is necessary for performing the medical task (the instrument head) is inserted between the marker attaching device (i.e. the adaptor) and the marker device. Also, the attaching part in this case is not obviously universally adaptable.

### SUMMARY OF THE INVENTION

It is an object of the invention to fasten, in this method, a marker device to at least one of the cited objects, in order to enable navigation even though a marker device is not integrated into at least one of the objects and/or a dedicated support is not provided on at least one of the objects.

The above object is solved by the subjects of the independent claims. The dependent claims are directed to advantageous developments of these subjects.

The marker attaching device in accordance with the invention is designed to attach a marker device to an object, in particular to a medical instrument and/or an anatomical body structure and/or an implant. The marker attaching device is preferably provided with a part for fastening a marker device and with an attaching part for attaching the marker attaching device to the object. The attaching part can preferably be deformed, in particular adapted or molded to at least one given shape of the object, such that the attaching device and/or attaching part enters into a mechanical connection with the object. The attaching part can in particular be deformed such that it abuts a surface of the object. A mechanical connection and/or a bond, for example a positive-fit connection between the attaching part and the object, can be assisted by the circumference of the attaching part along its surface which faces the object when it is attached being approximately equal to the circumference of at least one part of the object to which the attaching part is attached, such that the attaching part exactly encompasses at least one part of the object. This enables the attaching part to be universally adaptable to objects of different geometries. The attaching part thus establishes a connection, in particular a stationary connection, between a marker device and objects of any shape, even if the latter were not originally provided for being combined with a marker device. The attaching part is in this respect designed for a mechanical connection to the object. The attaching device in accordance with the invention thus in particular serves as an adaptor for attaching a marker device to a multitude of differently shaped objects. The attaching part can be attachable to the object by plastic deformation and possibly detachable from it. The fastening part can for example be a constituent of the attaching device, for example an edge, an extension or the periphery of a hole, wherein the marker device can be attached to the constituent. The fastening part can also include a mechanical holding connection for mechanically connecting the marker attaching device to the marker device, wherein said holding connection is joined to the attaching part and designed for a mechanical connection (for example, a force-fit and/or frictional-fit and/or material-fit and/or positive-fit, stationary and/or releasable connection) and/or a bond with the marker device and/or the attaching part and for example includes a snap-on connection, locking connection, screw connection, latch elements, threads, etc. Within the framework of this invention, the term “mechanical connection” and/or “bond” includes a force-fit and/or frictional-fit and/or positive-fit and/or material-fit connection.

The attaching part is preferably produced from a material which is mechanically rigid and/or firm in its raw form and/or when molded and which becomes more easily plastically deformable, i.e. using a lower application of force than in its raw form and/or when molded, when it is subjected to energy which can for example come from physical and/or chemical processes, in particular mechanical oscillations and/or waves and/or thermal energy and/or electromagnetic radiation and/or particular chemical reactions, i.e. when it is subjected to external energy or when external energy is released (i.e. it is more deformable than before the application of energy). The attaching part can be deformed by forces which can be achieved by a human hand without additional aids, i.e. for example by forces using one's bare hands which have a lower limit of about 0.1 N, 1 N, 10 N and/or an upper limit of about 10 N, 100 N, 1000 N. The attaching part can thus be attached quickly and easily without additional aids or particular technical knowledge, even under operating theatre conditions.

In accordance with one embodiment of the invention, the attaching part is produced from a material which in its raw form is easily plastically deformable, i.e. by forces which can be achieved by a human hand without additional aids, i.e. for example by forces using one's bare hands which have a lower limit of about 0.1 N, 1 N or 10 N and/or an upper limit of about 10 N, 100 N or 1000 N. When molded, the material of the attaching part is mechanically rigid and/or firm. It becomes more difficult to deform or impossible to deform, i.e. for example it assumes a harder degree of hardness, when it is subjected to energy which can for example include physical and/or chemical processes, in particular mechanical oscillations and/or waves and/or thermal energy and/or electromagnetic radiation and/or particular chemical reactions, i.e. when it is subjected to external energy or when external energy is released. This means it only becomes more plastically deformable by a greater application of force than in its raw form and/or before the application of energy, in particular an application of force which is above the aforementioned upper limits. A method which can be realized under operating theatre conditions can thus be used to achieve a firm hold using a mechanically stable attaching part.

The marker attaching device can be held on the object by a mechanical connection and/or a bond, in particular in a positive fit and/or material fit and/or force fit and/or frictional fit, such that it is stationary and/or detachable from the object.

The attaching part can preferably be deformed such that, once deformed, it at least partially surrounds the object in order to achieve the desired attachment. If the attaching part surrounds the object completely, then the surface of the attaching part is closed while the surface surrounds the object. Preferably, however, its surface is interrupted at least one point, such that a hollow space is created in the interior of the attaching part, in which the object is situated, wherein the hollow space has a connection to the surrounding atmosphere through the interruption in the surface. The attaching part thus has in particular at least one and preferably two outer edges which run in a closed loop and between which there extends an outer surface which points outwards (and when attached to the object, points away from the object) and an inner surface which points inwards (and when attached to the object, points towards the object). This preferred shape shall be referred to from here on in as the “open shape”, since it provides at least one opening which is defined by a closed outer edge and through which the object can protrude outwards (as viewed from the attaching part). In particular when there are two outer edges, these outer edges can also be interrupted such that the outer surface and the inner surface are also correspondingly interrupted. In order to bridge this continuous area interruption, the attaching part can include a closing mechanism such as is described below. The area interruption can facilitate introducing the object into a space surrounded by the inner surface. Due to the open shape, the part of the anatomical structure, medical instrument and/or implant necessary for the medical application can protrude from the part enclosed (at least partially) by the attaching part. The shape modeled from the attaching part can have a material thickness having a lower limit of for example 0.5 mm, 1 mm or 5 mm and/or an upper limit of for example 2 mm, 5 mm or 1 cm or 2 cm. The attaching part can also include a mechanical closing connection (for example a latch and/or snap fastening, a Velcro fastening, a popper fastening, a device which is similar or identical to a belt buckle, a threaded connection, a nail, a screw, a welded seam and/or a bonded seam, i.e. a positive-fit and/or force-fit and/or frictional-fit and/or material-fit connection which can be designed to be releasable and/or stationary) which assists in seating the marker attaching device on the object in a force fit and/or frictional fit and/or positive fit and/or material fit and/or in detaching the marker attaching device from the object. The attaching part preferably includes a slip-inhibiting means (which can for example be a material and/or a device and/or a chemical agent and can for example be formed by a protrusion from a surface of the attaching part and a material property of the attaching part) on for example its inner surface which in particular faces the object. The surface which, when the attaching part is attached, abuts and/or faces a surface of the object is referred to as its inner surface. At least one additional protrusion which in particular protrudes from one of the surfaces of the attaching part (for instance, a spike-shaped or wedge-shaped and/or rounded protrusion) and/or a slip-inhibiting surface which derives its slip-inhibiting property for example from an adhesion-enhancing and/or dynamic friction-enhancing coating (comprising for instance a rubber-like material, for example plasticised polyvinyl chloride (PVC) and/or vulcanized rubber) or a roughness-increasing treatment (for example by grinding or sandblasting) can assist a non-slip seating of the attaching part on the object. This can prevent an undesirable change in the position of the attaching part relative to the object during the operation.

The attaching part can consist of a number of parts. A first part of the attaching part can be formed from a different material to a second part of the attaching part which is different to the first part, wherein the material in the first part of the attaching part in particular has a greater specific strength than the material in the second part of the attaching part. It is thus possible for example to avoid mechanical damage to the attaching part during use by forming a part of the attaching part which is more heavily used, and for example includes the mechanical closing connection, from a harder and/or more resilient material or with a greater material thickness than in a second part. Forming a part of the attaching part which is provided for deformation from a more flexible and/or less hard material than for example in a part which is not provided for molding can also assist in making molding easier.

The marker device which is fastened to the marker attaching device can be produced from a metal and/or a plastic.

In the marker attaching device described above, a counter support which comprises a metal or a plastic can be provided on the surface of the attaching part opposite the surface of the attaching part on which the marker device is attached and/or towards which the marker device points. The counter support can be designed to be plate-shaped and/or disc-shaped, spherical or in the shape of a parallelepiped or polyhedron and can exhibit a material thickness having a lower limit of for example 0.5 mm, 1 mm or 2 mm and/or an upper limit of for example 2 mm, 5 mm or 1 cm. The surface of the counter support which assists in firmly seating the marker device on the attaching part can for example have a surface area having a lower limit of for example 5 mm² and/or an upper limit of for example 2 cm² or 10 cm². The counter support can also be produced from a flexible, elastic material (for instance, from a rubber or a plastic) and/or from a mechanically firm material (for example a metal, in the manner of a commercially available washer). The counter support is intended to prevent the marker device from slipping relative to the attaching part (for example by wobbling or being unintentionally touched).

The part for fastening the marker device to the marker attaching device can include a mechanical holding connection, wherein the mechanical holding connection in particular includes at least a positive-fit and/or force-fit and/or frictional-fit and/or material-fit connection. The mechanical holding connection for fastening the marker device to the marker attaching device can be embodied in the form of a latch mechanism, a threaded connection, a popper, a nail, a screw, a welded seam, a bonded seam and/or a Velcro fastening. A combination of these types of holding connection can also be used. The mechanical holding connection is preferably intended to act in a positive fit and/or force fit and/or frictional fit and/or material fit and stationary and/or to be releasable. The fastening part can also for example be formed by or include the periphery of a hole in a surface of the attaching part. The mechanical holding connection assists in the stationary seating of the marker device on the marker attaching device and can help to make the two parts easy to detach from each other.

The marker attaching device can be designed such that it can be attached to a medical instrument and/or an anatomical body structure and/or an implant. To this end, respectively matching combinations of materials have to be chosen in order to ensure a force-fit and/or frictional-fit and/or positive-fit seating. In the latter two cases, the physiological compatibility of the materials of the marker attaching device also has to be considered.

The marker attaching device can include a marker device which in turn includes a reference star and/or at least one marker element. This marker device can be fastened to the attaching part of the marker attaching device by means of the aforementioned fastening part.

The marker attaching device and a medical apparatus and/or an implant can form a system which is provided to the user as a package.

The marker attaching device and a marker device can also form a system which is provided to the user as a package.

A marker attaching device as described above can be used by attaching the attaching device to an object, in particular to a medical instrument and/or an anatomical body structure and/or an implant.

A method for fastening the marker attaching device described above to an object, in particular to a medical instrument and/or an anatomical body structure and/or an implant, can include the following sequence: firstly, the material of the attaching part is supplied with energy, in order for example to heat it, wherein energy from for example mechanical oscillations and/or waves and/or thermal energy and/or electromagnetic radiation and/or a chemical reaction and/or electric current (i.e. energy from physical and/or chemical processes) acts on the material of the attaching part, in order to make the material of the attaching part more easily plastically deformable than before the application of energy. The attaching part can in particular be impossible to plastically deform, i.e. hard, before the application of energy. The attaching part thus becomes more plastically deformable by forces which can be achieved by a human hand without additional aids, i.e. for example by forces using one's bare hands which have a lower limit of about 0.1 N, 1 N or 10 N and/or an upper limit of about 10 N, 100 N or 1000 N. The attaching part is then molded to the structure (i.e. the outer shape) of the object. Lastly, the material of the attaching part can harden, i.e. can be made harder. The following sequence is also possible: the material of the attaching part is provided to the user in a raw form which comprises the material in a plastically deformable state (i.e. in a deformation state). In this state, the material of the attaching part can be deformed by forces which can be achieved by a human hand without additional aids, i.e. for example by forces using one's bare hands which have a lower limit of about 0.1 N, 1 N or 10 N and/or an upper limit of about 10 N, 100 N or 1000 N. The attaching part is then molded to the structure (i.e. the outer shape) of the object. Lastly, the material of the attaching part is supplied with energy, in order for example to heat it, wherein for example mechanical oscillations and/or waves and/or thermal energy and/or electromagnetic radiation and/or a chemical reaction and/or electric current (i.e. energy from physical and/or chemical processes) act on the material of the attaching part, in order to harden it, i.e. transfer it into a mechanically firmer, in particular harder and more resilient state (the application state). The statement with respect to the above methods that the material of the attaching part becomes plastically deformable by being subjected to energy includes the statement that the material of the attaching part is more plastically deformable (i.e. more easily deformable and/or deformable using a lower application of force) in its deformation state than before the application of energy. The material of the attaching part can in particular achieve a higher tensile strength and/or compressive strength and/or shearing strength in its application state than in its raw-form state and/or deformation state. The material of the attaching part can also be mechanically harder in its application state than in its raw-form state and/or deformation state.

A method for removing the marker attaching device described above from an object, in particular from a medical instrument and/or an anatomical body structure and/or an implant, can include the following sequence: firstly, the material of the attaching part is supplied with energy, in order for example to heat it, wherein for example mechanical oscillations and/or waves and/or thermal energy and/or electromagnetic radiation and/or a chemical reaction and/or electric current (i.e. energy from physical and/or chemical processes) act on the material of the attaching part, in order to make the attaching part more easily deformable than before the application of energy. The material of the attaching part becomes plastically deformable by forces which can be achieved by a human hand without additional aids, i.e. for example by forces using one's bare hands which have a lower limit of about 0.1 N, 1 N or 10 N and/or an upper limit of about 10 N, 100 N or 1000 N. Lastly, the attaching part can be spatially removed from the object using for example a separating means (for example, a pair of scissors or a knife or a pair of forceps).

Other advantages and features of the invention are disclosed in the following detailed description. Features of different embodiments can be combined with each other.

The attaching part advantageously ensures that the marker device is firmly seated on the object during all the processing steps in the operation. Advantageously, it should also for example have properties which ensure that it is perfectly hygienic, for example resilience to chemical disinfectants and/or cleaning methods (for example heating) which are usually used. The attaching part can alternatively be produced as a disposable item which can be disposed of once it has been used. It can for example be produced from a plastic and/or a metallic sheet, wherein these materials can be integrated into conventional disposal systems without any problems.

The material from which the attaching part is produced should also be able to withstand the chemical and mechanical demands on it during an operation. This can be achieved by using a plastic which for example includes aramid-containing fibers.

Advantageously, the attaching part can be universally adapted to different surfaces, in order that it can be attached to various medical instruments. The attaching part is advantageously provided to the user in a raw form which the user can then mould to the surface of the instrument in question, wherein the attaching part passes from a deformation state into an application state. The attaching part can advantageously be deformed by forces which can be achieved by a human hand without additional aids, i.e. for example by forces using one's bare hands which have a lower limit of about 0.1 N, 1 N or 10 N and/or an upper limit of about 10 N, 100 N or 1000 N, in order to ensure easy handling.

In order to broaden the applicability of the attaching part, the attaching part can be designed such that it can be attached not only to medical instruments but also to anatomical body structures, wherein—in addition to the properties of the object in accordance with the invention as required above—particularly easy handling, compatibility with the human body and avoiding injury to the patient and user should be considered. This can be achieved by using biocompatible and/or physiologically compatible polymers in the material of the attaching part, such as for instance silicone polymers.

Advantageously, the attaching part can be designed such that it can be reabsorbed by the human body, such that it is not necessary to remove it after the end of the operation. This can be achieved by using biocompatible polymers which can be reabsorbed in the material of the attaching part, such as for instance polylactide, polyglycolide (generally, polymers based on lactic acid and/or glycolic acid).

### BRIEF DESCRIPTION OF THE DRAWINGS

FIG. 1 shows the molding process of a marker attaching device in accordance with the invention, comprising a tube-shaped attaching part and a part for fastening a marker device to a medical instrument.

FIG. 2 shows the marker attaching device in accordance with the invention in the form of a belt, comprising a closing device in the form of a latch fastening.

FIG. 3 shows the marker attaching device in accordance with the invention, comprising an attaching part in the form of a belt comprising a closing device in the form of a latch fastening, and comprising a slip-inhibiting coating on the surface of the attaching part which faces the object.

FIG. 4 shows the marker attaching device in accordance with the invention, comprising an attaching part in the form of a belt, comprising a closing device in the form of a latch fastening and a mechanical holding connection in the form of a fixing screw.

FIG. 5 shows the marker attaching device in accordance with the invention, comprising an attaching part which is at least partially designed in the form of a net and a closing device in the form of a latch fastening.

FIGS. 6a and 6b show the marker attaching device in accordance with the invention, comprising an attaching part and a closing device in the form of a latch fastening, and different embodiments of a slip-preventing means for assisting in a non-slip seating of the attaching part.

FIG. 7 shows the marker attaching device in accordance with the invention, and how it can for example be fastened to an anatomical body structure or an implant.

FIG. 8 shows the marker attaching device in accordance with the invention, with a marker device attached to it.

FIG. 9 shows the marker attaching device in accordance with the invention, with an individual marker element attached to it.

FIG. 10 shows the marker attaching device in accordance with the invention, comprising a counter support for stabilizing a marker device which is connected to the marker attaching device.

FIG. 11 shows a first method sequence for fastening an attaching part of a marker attaching device to an object.

FIG. 12 shows a second method sequence for fastening an attaching part of a marker attaching device to an object.

FIG. 13 shows a third method sequence for fastening an attaching part of a marker attaching device to an object.

FIG. 14 shows a fourth method sequence for fastening an attaching part of a marker attaching device to an object.

FIG. 15 shows a method sequence for removing an attaching part of a marker attaching device from an object.

### DETAILED DESCRIPTION

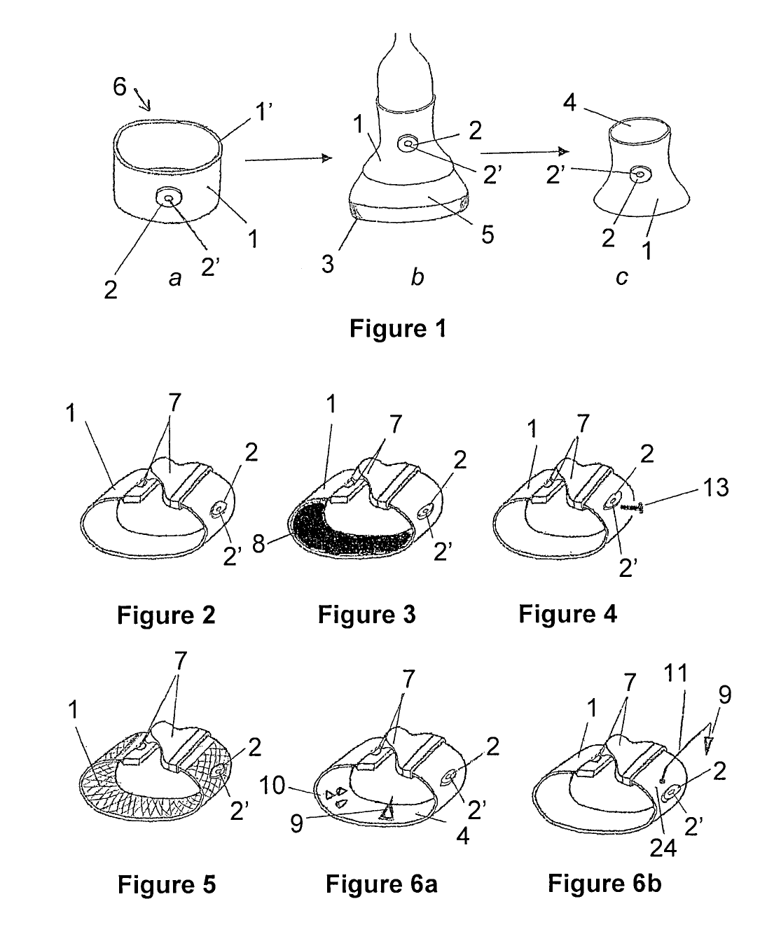

The attaching part 1 can for example be provided to the user in a raw form which is provided for being molded to the object 3, 14. An application shape (in which the attaching part 1 is in an application state) is created from the raw form and holds the attaching part 1 on the object 3, 14. This raw form can advantageously be embodied in the form shape of a tube, as in FIG. 1a. It can however also for example have a raw form comprising an open area, such as a toroid or a belt (as in FIGS. 2 to 4) or a cuboid and/or spherical and/or ellipsoid clump of material. In one embodiment of the invention, the material of the attaching part 1 can be provided in a mechanically firm state or also in a deformable state (i.e. a deformation state) in its raw form.

FIG. 1 shows how a tube-shaped attaching part 1 in accordance with the invention, comprising a part 2 for fastening a marker device, is slid over an object—in this case, a conical medical instrument 3 or an anatomical body part and/or an implant 14 (cf. FIGS. 1, 7 and 11 [reference sign S1]). The attaching part 1 can in particular consist of one piece, but can also consist of a number of pieces (i.e. at least two pieces). The attaching part 1 is then placed in a plastically deformable state (deformation state) by being subjected to energy (S2); the attaching part 1 can then be deformed (S3) such that its surface 4 which points towards the surface 5 of the object 3 can be adapted to the surface 5 of the object 3 (S4). This establishes a positive-fit seating of the attaching part 1 on the object 3. The attaching part 1 can also however have an open shape in its application shape, in which the two outer edges are interrupted. In order to bridge this continuous area interruption, the attaching part can include a closing mechanism such as is described below. The area interruption can facilitate introducing the object into a space surrounded by the inner surface. The shape gives the attaching part 1 the shape of a clip comprising a positive-fit and/or force-fit and/or frictional-fit hold on the object 3. Lastly, the material of the attaching part 1 has to harden, i.e. be made harder and/or firmer (S5), which can for example be achieved by drying it in the ambient air or by an assisting treatment using heated or cooled air from a blow-dryer and/or fan.

The material of the attaching part 1 can for example be made deformable by producing the attaching part 1 from a material which becomes plastically deformable, i.e. is placed in a deformation state, when it is subjected to acoustic oscillations and waves and/or mechanical shearing and/or shearing oscillations and waves. To this end, the material of the attaching part 1 has to be subjected to mechanical stress using the corresponding oscillations and/or waves before it is adapted to a given surface of an object. Materials of the attaching part 1 which may be considered for this approach include thermoplastics such as for example polyamides and/or polycarbonates and/or polyethylene therephthalate and/or polyethylene and/or polypropylene and/or polystyrene and/or polyvinyl chloride.

The material of the attaching part 1 can for example be made deformable by including a material (such as for instance polyethylene and/or polycarbonate and/or polyamides and/or polyester and/or polyacrylates) which becomes plastically deformable when it is subjected to ultrasound and/or infrasound waves. To this end, the material of the attaching part 1 has to be irradiated with ultrasound and infrasound waves of a suitable intensity and for a suitable duration before it is adapted to a given surface 5 of an object 3. Other materials of the attaching part 1 which may be considered for this method include thermoplastics such as for example polyamides and/or polycarbonates and/or polyethylene therephthalate and/or polyethylene and/or polypropylene and/or polystyrene and/or polyvinyl chloride.

In one embodiment of the invention, the material of the attaching part 1 can be made deformable by including a material which becomes plastically deformable when it is subjected to thermal energy. To this end, the material of the attaching part 1 has to be exposed to the influence of thermal energy, i.e. heated to a suitable temperature using a suitable quantity of energy, before the attaching part 1 is adapted to the object 3. The attaching part 1 can then be plastically deformed and adapted to a given surface 5. Given these materials, heating to a temperature in a range having a lower limit of 30° C. or 40° C. or 50° C. and an upper limit of 190° C. or 200° C. or 210° C. and/or which is for example about 60° C. or 100° C. or 150° C. can be sufficient in order to make the material of the attaching part 1 plastically deformable.

In another embodiment of the invention, the material of the attaching part 1 can be made deformable by including a material which becomes plastically deformable when it is subjected to infrared radiation. To this end, the material of the attaching part 1 has to be exposed to the influence of infrared radiation before the attaching part 1 is adapted to a given surface 5. This can for example be achieved by irradiating it with a light source which emits electromagnetic radiation in the infrared range. The attaching part 1 can then be molded to a given surface 5 of an object 3. Materials of the attaching part 1 which may be considered for this approach include thermoplastics such as for example polyamides and/or polycarbonates and/or polyethylene therephthalate and/or polyethylene and/or polypropylene and/or polystyrene and/or polyvinyl chloride.

In another embodiment of the invention, the material of the attaching part 1 can be made deformable by including a material which becomes plastically deformable when it is subjected to microwave radiation. To this end, the material of the attaching part 1 has to be exposed to the influence of microwave radiation before the attaching part 1 is adapted to a given surface 5. This can for example be achieved by placing the material of the attaching part 1 in a commercially available microwave oven and irradiating it in said oven with microwave radiation. The attaching part 1 can then be adapted to a given surface 5. Materials of the attaching part 1 which may be considered for this approach include thermoplastics such as for example polyamides and/or polycarbonates and/or polyethylene therephthalate and/or polyethylene and/or polypropylene and/or polystyrene and/or polyvinyl chloride.

In another embodiment of the invention, the material of the attaching part 1 can be made deformable by including a material which becomes plastically deformable when it is subjected to ultraviolet radiation. To this end, the material of the attaching part 1 can for example be irradiated with a light source which emits electromagnetic radiation in the ultraviolet spectral range. The attaching part 1 can then be deformed and adapted to a given surface 5.

The material of the attaching part 1 can also for example be made deformable by including a material which becomes plastically deformable when it is subjected to visible light. To this end, the material can for example be irradiated with a light source which emits electromagnetic radiation in the visible spectral range. The attaching part 1 can then be deformed and adapted to a given surface. Materials of the attaching part 1 which may be considered for this approach include thermoplastics such as for example polyamides and/or polycarbonates and/or polyethylene therephthalate and/or polyethylene and/or polypropylene and/or polystyrene and/or polyvinyl chloride.

In another embodiment, the material of the attaching part 1 can be made deformable by including a material which becomes plastically deformable when it is subjected to particular chemical reactions. To this end, the material of the attaching part 1 can for example be exposed to an external treatment using a chemical compound, which is for instance performed by way of a bath in a liquid or gaseous chemical compound. Alternatively, the material of the attaching part 1 can be designed such that it is constructed from different components which contain particular chemical compounds which can be mixed with each other, for example by kneading, in the manner of a solid two-component adhesive (for example, methyl methacrylate adhesive) and so enter into a chemical reaction which makes the material of the attaching part 1 plastically deformable. This can also for example be effected by an exothermic chemical reaction, such that the thermal energy released by the reaction makes the material of the attaching part 1 plastically deformable.

The statement with respect to the above embodiments that the material of the attaching part 1 becomes plastically deformable by being subjected to energy includes the statement that the material of the attaching part 1 is more plastically deformable (i.e. more easily deformable and/or deformable using a lower application of force) in its deformation state than before the application of energy. The material of the attaching part 1 can in particular achieve a higher tensile strength and/or compressive strength and/or shearing strength in its application state than in its raw-form state and/or deformation state. The material of the attaching part 1 can also be mechanically harder in its application state than in its raw-form state and/or deformation state.

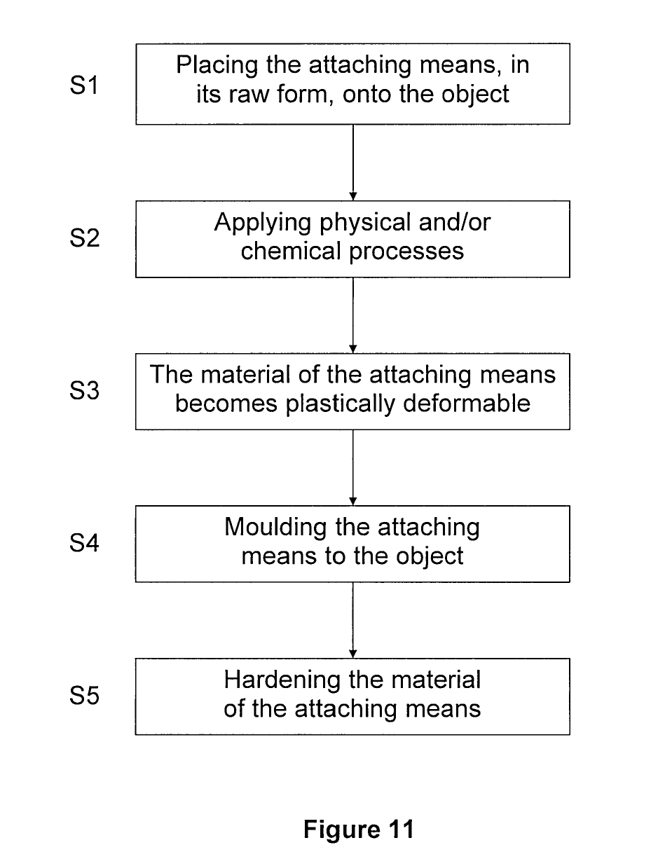

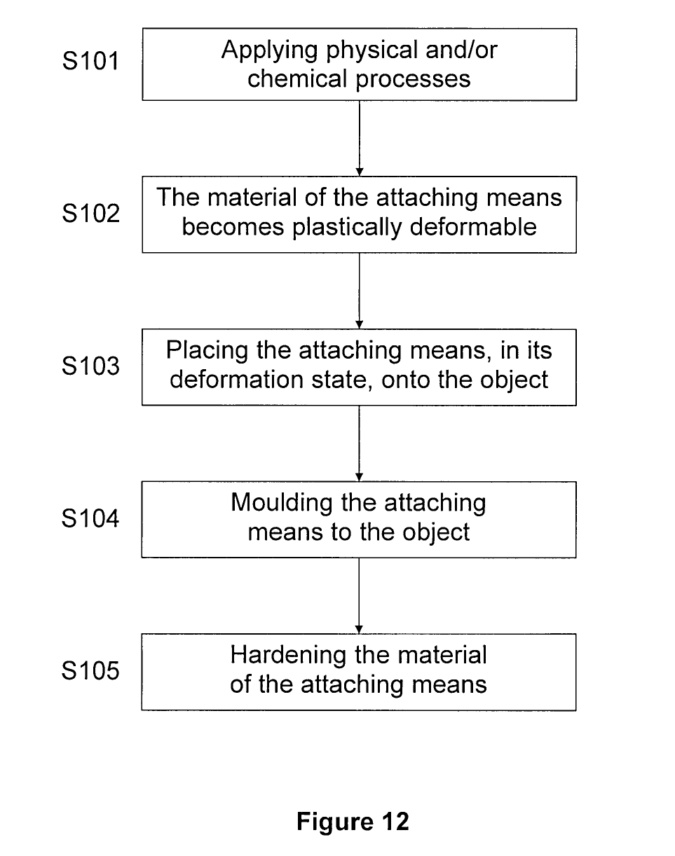

As applicable, the attaching part 1 can—as shown in FIG. 11—be placed onto the given surface 5 of an object 3 in its raw form (S1), in order to only then be made plastically deformable (i.e. placed in its deformation state) using one of the aforementioned methods (S2, S3) and then molded to the surface 5 as desired (S4). To this end, however, the surface and/or object has to be such that it withstands the physical and/or chemical influences of the method chosen. As shown in FIG. 12, the process can optionally also be performed such that the attaching part 1 is placed (S102), spatially separate from the surface 5 of the object 3, in a plastically deformable state (i.e. in a deformation state) in accordance with one of the aforementioned methods (S101) and only then placed (S103) onto the object 3 and molded (S104). This approach has the advantage that a surface 5 of the object 3 (in particular, a surface of a medical instrument and/or anatomical structure), which does not withstand increased chemical demands—in accordance with the method of FIG. 11—as compared to its standard use, remains unimpaired.

Once the attaching part 1 has been molded (S4, S104) to the given surface 5, the material of the attaching part 1 is preferably hardened and/or made mechanically firmer (S5, S105), i.e. placed in an application shape and/or application state, in order to achieve the mechanical strength required in order to stably seat the marker attaching device 6 on the object 3, 14.

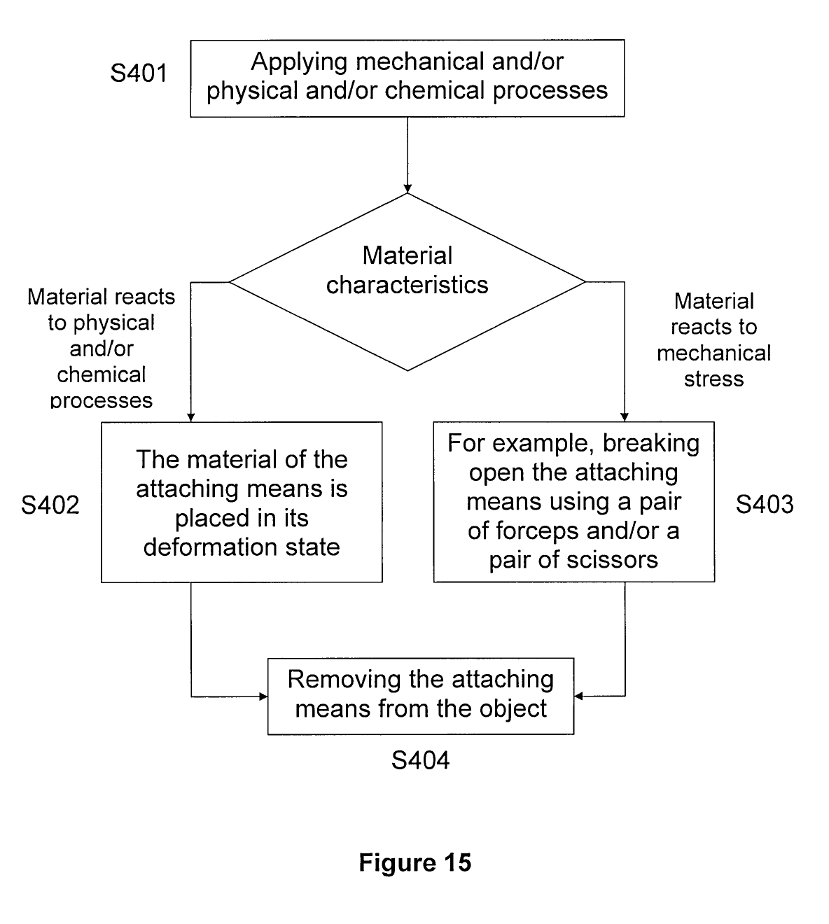

As shown in FIG. 15, the attaching part 1 can, as applicable, be removed (S404) from the object 3, 14 mechanically, for example with the aid of a separating device, for example a pair of scissors or a knife or a pair of forceps (S403). At least one of the physical-chemical methods (S2, S101, S401) which can be used to transfer the material of the attaching part 1 from its solid raw form into a deformation state (S3, S102, S402) is in particular used for removing the attaching part 1 from the object 3, 14, which then allows the attaching part 1 to be easily detached from the object 3, 14.

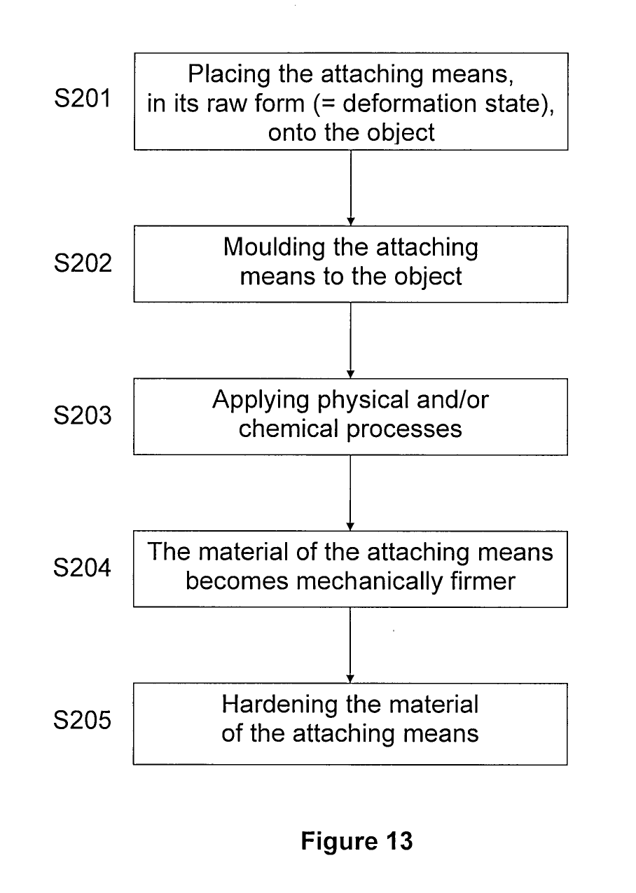

However, in yet another embodiment—as shown in FIG. 13—it is also possible for the attaching part 1 to be provided in a deformation state (S201) as its raw form even before it is slid over the medical instrument 3, and to be slid over the medical instrument 3 and molded without the user first having to make it deformable (cf. the method sequence in FIG. 13). It can then be hardened and/or solidified (S204, S205), for example by being air-dried: to this end, the material need only dry at room temperature in the ambient air; preferably, however, the hardening process can also be assisted by an air blow-dryer using air which may be heated or cooled as compared to the ambient air, in order to give the material the desired strength.

The material of the attaching part 1 can for example be hardened and/or solidified (S204, S205) by including a material which becomes mechanically firm when it is subjected (S203) to acoustic oscillations and waves and/or mechanical shearing and/or shearing oscillations and waves. To this end, the material of the attaching part 1 has to be subjected to mechanical stress using the corresponding oscillations and/or waves after it is adapted to a given surface of an object.

The material of the attaching part 1 can for example be hardened and/or solidified (S204, S205) by including a material which becomes mechanically firm when it is subjected (S203) to ultrasound and/or infrasound waves. To this end, the material of the attaching part 1 has to be irradiated with ultrasound and infrasound waves of a suitable intensity and for a suitable duration after it is adapted to a given surface 5 of an object 3.

In one embodiment of the invention, the material of the attaching part 1 can be hardened and/or solidified (S204, S205) by including a material which becomes mechanically firm when it is subjected (S203) to thermal energy. To this end, the material of the attaching part 1 has to be exposed to the influence of thermal energy, i.e. heated or cooled to a suitable temperature using a suitable quantity of energy, after the attaching part 1 is adapted to the object 3.

In another embodiment of the invention, the material of the attaching part 1 can be hardened and/or solidified (S204, S205) by including a material which becomes mechanically firm when it is subjected (S203) to infrared radiation. To this end, the material of the attaching part 1 has to be exposed to the influence of infrared radiation after the attaching part 1 is adapted to a given surface 5. This can for example be achieved by irradiating it with a light source which emits electromagnetic radiation in the infrared range.

In another embodiment of the invention, the material of the attaching part 1 can be hardened and/or solidified (S204, S205) by including a material which becomes mechanically firm when it is subjected (S203) to microwave radiation. To this end, the material of the attaching part 1 has to be exposed to the influence of microwave radiation after the attaching part 1 is adapted to a given surface 5. This can for example be achieved by placing the material of the attaching part 1 in a commercially available microwave oven and irradiating it in said oven with microwave radiation.

In another embodiment of the invention, the material of the attaching part 1 can be hardened and/or solidified (S204, S205) by including a material which becomes mechanically firm when it is subjected (S203) to ultraviolet radiation. To this end, the material of the attaching part 1 can for example be irradiated with a light source which emits electromagnetic radiation in the ultraviolet spectral range.

The material of the attaching part 1 can advantageously be hardened and/or solidified (S204, S205) by including a material which becomes mechanically firm when it is subjected (S203) to visible light. To this end, the material can for example be irradiated with a light source which emits electromagnetic radiation in the visible spectral range.

The material of the attaching part 1 can advantageously be hardened and/or solidified (S204, S205) by including a material which becomes firm when it is subjected (S203) to particular chemical reactions. To this end, the material of the attaching part 1 can for example be exposed to an external treatment using a chemical compound, which is for instance performed by way of a bath in a liquid or gaseous chemical compound. Alternatively, the material of the attaching part 1 can be designed such that it is constructed from different components which contain particular chemical compounds which can be mixed with each other, for example by kneading, in the manner of a two-component adhesive (for example, methyl methacrylate adhesive) which can be plastically deformed by human forces, and so enter into a chemical reaction which makes the material of the attaching part 1 mechanically firm. This can also for example be effected by an exothermic and/or endothermic chemical reaction, such that the thermal energy released by the reaction or taken from the environment by the reaction makes the material of the attaching part 1 mechanically firmer (i.e. firmer than before the reaction).

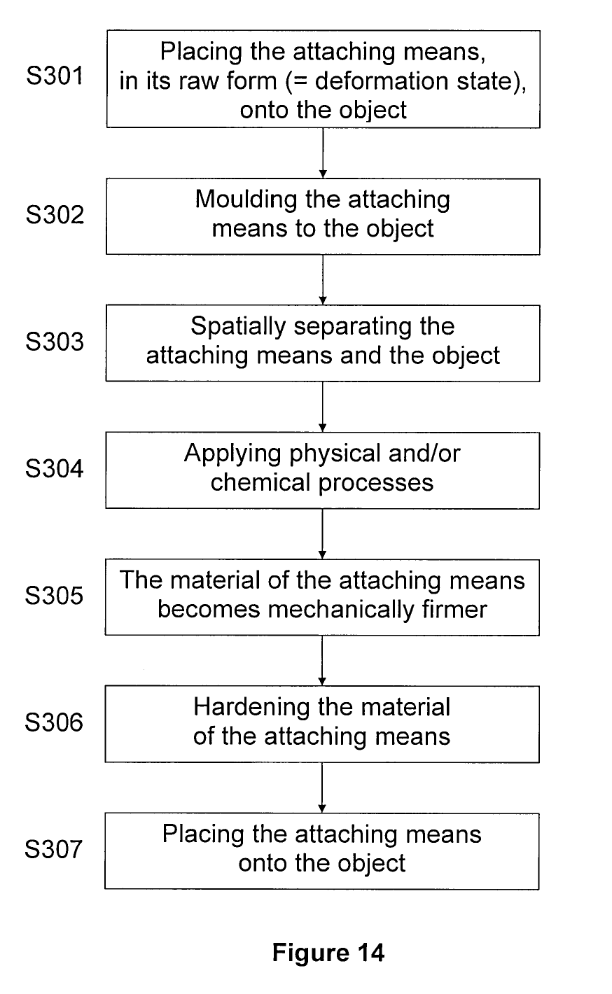

In this embodiment, the attaching part 1 can—as shown in FIG. 14—be placed (S201) onto the given surface 5 of an object 3, 14 in its raw form and then molded (S202) to the surface 5 as desired, in order to only then be made mechanically firm using one of the aforementioned methods. To this end, however, the surface 5 and/or object 3, 14 has to be such that it withstands the physical and/or chemical influences of the method chosen. The process can optionally also be performed such that the attaching part 1 is placed (S301) onto the object 3, 14 and deformed (S302) first, and then—spatially separate (S303) from the surface 5 of the object 3—placed in a firm state (S305) in accordance with one of the aforementioned methods (S304) and, once hardened and/or solidified (S306), is placed onto the object 3, 14 again (S307). This approach has the advantage that a surface 5 of the object 3 (in particular, a surface of a medical instrument and/or anatomical structure), which does not withstand increased chemical demands—in accordance with the method of FIG. 11—as compared to its standard use, remains unimpaired.

As shown in FIG. 15, the attaching part 1 can, as applicable, be removed (S404) from the object 3, 14 mechanically, for example with the aid of a separating means, for example in the form of a pair of scissors or a pair of forceps (S403). At least one of the physical-chemical methods (S2, S101, S401) which can be used for hardening and/or solidifying (S204, S205, S305, S306) is preferably used to place the material of the attaching part 1 in a deformation state (i.e. in its raw form) again, which then allows the attaching part 1 to be easily detached (S404) manually from the object 3, 14.

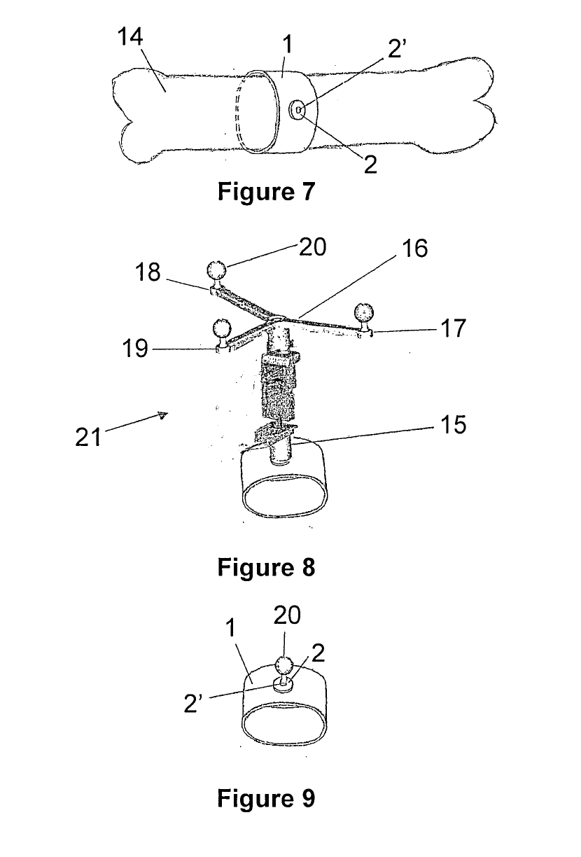

As can be seen from FIG. 1, the attaching part 1 is in this case held on the object 3, 14 in a positive fit. The marker attaching device 6 can advantageously have an attaching part 1 in the form of a surface area as in FIG. 1, for example in the form of a tube-shaped and/or cylindrical sleeve, or an open area such as is defined further above. The attaching part 1 can also, however, be embodied in the form of a belt or ring, as in FIG. 2. The attaching part 1 is preferably at least partially designed in the form of a net, as in FIG. 5. As indicated in FIGS. 2 to 4, the attaching part 1 can also include a closing device 7 (for example, a latch fastening and/or snap fastening, a Velcro fastening, a popper fastening, a device which is similar or identical to a belt buckle, a threaded connection, a nail, a screw, a welded seam and/or a bonded seam) which assists in seating the marker attaching device 6 on the object 3 in a force fit and/or frictional fit and/or positive fit and connects at least two open ends of the attaching part 1 to each other (for example, in a surface area). This enables an additional mechanical tension, which serves to seat the marker attaching device 6 on an object 3 in a force fit and/or frictional fit, to be applied to the attaching part 1 when the attaching part 1 is being adapted to the given surface 5 of an object 3. A non-slip seating of the attaching part 1 on the object 3 can be assisted by providing the material of the attaching part 1 on the surface 4 of the attaching part 1 which directly abuts the given surface 5 of an object 3 with a slip-inhibiting coating 8. This slip-inhibiting coating 8 can in particular be designed such that it takes into account the surface characteristics of the object 3 to which the marker attaching device 6 is to be attached. The slip-inhibiting layer 8 can thus be adapted to the coefficients of dynamic and static friction of the given surface 5, its chemical characteristics (for example in order to avoid an undesirable reaction between the given surface 5 and the slip-inhibiting coating 8) and/or to the medical, physiological and/or hygiene requirements of the given surface 5 if the object 3 represents an anatomical body structure 14 and/or an implant 14 (as in FIG. 7). The slip-inhibiting layer 8 in particular increases the static friction between the surface 5 and the attaching part 1 as compared to an embodiment in which the slip-inhibiting layer 8 is not used. A non-slip seating of the attaching part 1 can also be assisted if the marker attaching device 6 includes a slip-preventing means, for example a protrusion 9 (as shown in FIGS. 4, 6a and 6b) which protrudes from one of the surfaces of the attaching part 1 (for instance, a spike-shaped or wedge-shaped and/or rounded protrusion), a nail or a screw 13, which protrudes from one of the surfaces 4, 24 of the attaching part 1 and ensures a firm connection between the marker attaching device 6 and the object 3 by countersinking and/or screwing the nail and/or screw 13 into the object 3 and/or the surface 5 of the object. Preferably, a multitude 10 of protrusions can also be used, wherein the protrusion 9 which protrudes from one of the surfaces of the attaching part 1 (for instance, a spike-shaped or wedge-shaped and/or rounded protrusion) can be connected to the attaching part 1 directly (as in FIG. 6a) or via an intermediate piece 11 (as in FIG. 6b). Advantageously, a non-slip seating of the attaching part 1 can be assisted by using a roughened surface 4.

In another embodiment of the invention, at least one region and/or peripheral region 1′ of the attaching part 1 can include a material which is different to the material which at least one other region of the attaching part 1 includes, wherein “another region” is in particular understood to mean a region which is situated away from the peripheral region 1′ (for example, with respect to the dimensions of the attaching part 1). The peripheral region 1′ can also include the region of the attaching part 1 on which the closing device 7 is situated and/or to which the closing device 7 is joined. The material in the peripheral region 1′ of the attaching part 1 can in particular be less deformable and/or less easy, i.e. more difficult, to adapt (requiring a greater application of force) to at least one given surface 5 than the material in at least one other region of the attaching part 1, given the same external conditions. The material in the peripheral region 1′ of the attaching part 1 can for example be 5%, 10%, 100% or 200% harder than the material in another region of the attaching part 1. This can for example serve the purpose that a material in the peripheral region 1′ of the attaching part 1 which is harder in its raw form and/or when molded prevents damage to the attaching part 1 and/or the object 3 during the adapting process or during the ongoing operation. During the adapting process, this can also prevent a shape of the attaching part 1 which is unfavorable for seating the marker attaching device 6 on the object 3 in a force fit and/or frictional fit and/or positive fit from being plastically modeled. The material in the peripheral region 1′ can assist in producing particular surface shapes of the attaching part 1 when adapting it to the object 3, 14 if, when it is plastically deformable, the material of the attaching part 1 includes at least one peripheral region 1′ comprising a predetermined structure which cannot be modeled and at least one other region of the attaching part 1 which can be modeled. The material in the peripheral region 1′ can also for example be 10%, 50% or 100% thicker than the material in at least one other region of the attaching part 1. This makes the attaching part 1 as a whole more rigid and more stable. As shown in FIG. 8, a marker device 21 which is provided for connecting to the marker attaching device 6 in accordance with the invention consists of a connector piece 15 and a multiple-arm holder 16 on which three adaptors 17, 18, 19 for marker elements 20 are situated, wherein said marker elements 20 can for example reflect electromagnetic radiation and thus enable the operation to be navigated within the framework of image-guided surgery (IGS), wherein each constituent of such a marker device 21 can be produced from a metal and/or a plastic. Particular care must therefore be taken when connecting the marker device 20 to the marker attaching device 6 in accordance with the invention that the mechanical properties of the materials of the marker device 21 and the marker attaching device 6 are such that they do not damage each other when subjected to mechanical stress. To this end, materials are preferably used in the marker device 21 and the marker attaching device 6, at least at a point of contact between the marker device 21 and the marker attaching device 6, which have the same hardness or the same order of magnitude of hardness. Care should also be taken that the materials do not influence each other chemically (for example, by an electrochemical reaction if the two materials are produced from different metals).

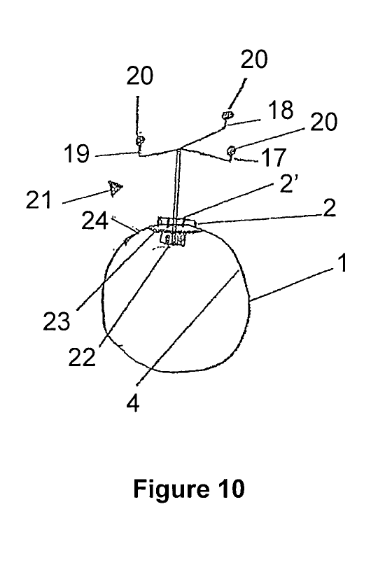

FIG. 10 shows how the seating of the marker device on the marker attaching device can be stabilized: the wobbling of the marker device 21 can for example be reduced by attaching a counter support (for example, a screw nut 21 together with a washer 23) on the surface 4 of the marker attaching device which is opposite the surface 24 of the marker attaching device on which the marker device 21 is attached and/or which points towards the marker device 21. This can also counteract a movement of the marker device 21 in a direction perpendicular to the area piece of the attaching part 1, to which the marker device 21 is attached. The counter support can for example be formed from metal or a plastic and can be embodied to be plate-shaped and/or embodied in the form of a commercially available washer and/or embodied to be disc-shaped or spherical or embodied in the form of a parallelepiped or a polyhedron or a nut and/or screw nut and can exhibit a material thickness having a lower limit of 0.5 mm or 1 mm or 2 mm and/or an upper limit of 2 mm or 5 mm or 1 cm, but should be connected (for example, such that it is stationary or releasable) to the connector piece 15 (for example by a screw thread or a material-fit connection, for instance an adhesive connection). The surface of the counter support which assists in firmly seating the marker device 21 on the attaching part 1 can for example have a surface area having a lower limit of for example 4 mm² or 10 mm² or 1 cm² and/or an upper limit of for example 1 cm² or 10 cm² or 40 cm². The counter support can also be produced from a flexible and/or elastic material (for instance, from a rubber or a plastic) and/or a mechanically firm material (for example a metal, in the manner of a commercially available washer).

The firm connection between the marker device 21 and the marker attaching device 6 can be produced by means of a mechanical holding connection 2′ included in the fastening part 2 and/or by a force-fit and/or frictional-fit and/or positive-fit and/or material-fit connection. Such a mechanical holding connection 2′ and/or such a force-fit and/or frictional-fit and/or positive-fit and/or material-fit connection can include a rivet such as in a commercially available popper fastening, a nail, a screw, a welded seam, a bonded seam, a Velcro fastening and/or a latching mechanism, as in FIGS. 2 to 6b. Preferably, a combination of these mechanical holding connections and/or types of connection can be used. The mechanical holding connection 2′ is at any rate intended to act in a positive fit and/or force fit and/or frictional fit and/or material fit and in particular stationary and in particular to be releasable. The fastening part can also for example include the periphery of a hole in a surface of the attaching part 1.

FIG. 7 shows a marker attaching device 6 which can be attached to an anatomical body structure 14 or an implant 14. In the latter case, care should be taken that it can be easily molded to the anatomical body structure 14 and/or implant 14 in its deformation state and that the marker attaching device 6 is seated in a force fit and/or frictional fit and/or positive fit such that no avoidable damage is caused to the anatomical body structure 14. To this end, and instead of the aforementioned protruding protrusion 9, 10, a non-slip seating of the attaching part 1 on the anatomical body structure 14 and/or implant 14 is preferably produced by an adhesive, in particular a physiologically compatible adhesive (which for example includes silicone polymers), i.e. in a material fit, wherein the adhesive is introduced between the surface 4 of the attaching part 1 facing the body structure 14 and/or implant 14 and the body structure 14 and/or implant 14 itself. In the interests of rationalizing the operation procedure and avoiding additional incisions, the marker attaching device 6 can also be designed such that it consists of a material which can be reabsorbed by the human and/or animal body (for example, polylactides and/or polyglycolide). The marker attaching device 6 can then remain on an anatomical body structure 14 and/or implant after the end of the operation, without having to be removed.

It is for example not only possible to attach a marker device 21, which represents a support for a multitude of markers 20, to the marker attaching device 6. Rather, it is also possible—as in FIG. 9—to fasten the marker device, in the form of an individual marker 20, to the marker attaching device 6. A mechanical holding connection 2′ on the fastening part 2, such as has been proposed further above, can be used for the connection which is then to be established between the marker 20 and the marker attaching device 6.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiment(s).

Although the invention has been shown and described with respect to one or more particular preferred embodiments, it is clear that equivalent amendments or modifications will occur to the person skilled in the art when reading and interpreting the text and enclosed drawing(s) of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the element described, i.e. which is functionally equivalent to it, even if it is not structurally equivalent to the disclosed structure which performs the function in the example embodiment(s) illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. A marker attaching device for attaching a marker device to an object, comprising a part for fastening a marker device and comprising an attaching part for attaching the marker attaching device, wherein the attaching part can be deformed in order to enter into a mechanical connection with the object.

2. The marker attaching device according to claim 1, wherein the object is a medical instrument and/or an anatomical body structure and/or an implant.

3. The marker attaching device according to claim 1, wherein the attaching part is produced from a material which becomes more plastically deformable by being supplied with energy.

4. The marker attaching device according to claim 1, wherein the attaching part includes a closing device for assisting in seating the marker attaching device on the object and/or at least one slip-inhibiting means for assisting in seating the attaching part on the object.

5. The marker attaching device according to claim 1, wherein a peripheral region of the attaching part is formed from a different material and/or is designed to be thicker than at least one other region of the attaching part, wherein the material in the peripheral region of the attaching part is more difficult to plastically deform (i.e. requiring a greater application of force) than the material in at least one other region of the attaching part.

6. The marker attaching device according to claim 5, wherein the different material is a harder material.

7. The marker attaching device according to claim 1, wherein a counter support is provided on the surface of the attaching part opposite the surface of the attaching part which the marker device points towards.

8. The marker attaching device according to claim 1, wherein the part for fastening the marker device to the marker attaching device includes a mechanical holding connection for mechanically connecting the marker attaching device to the marker device.

9. A system consisting of a marker attaching device according to claim 1 and a medical apparatus and/or an implant which is connected to the marker attaching device.

10. A system consisting of a marker attaching device according to claim 1, or the system according to claim 9, and a marker device.

11. A method for fastening a marker attaching device according to claim 1 to an object, comprising at least one of the following steps:
subjecting the material of the attaching part to energy, in order to make the material of the attaching part more plastically deformable;
molding the attaching part to at least one given surface of the object.

12. The method according to claim 11, wherein the object is a medical instrument and/or an anatomical body structure and/or an implant.

13. The method according to claim 11, wherein it also includes the following step: making the attaching part firmer.

14. A method for fastening a marker attaching device according to claim 1 to an object, comprising at least one of the following steps:
subjecting the material of the attaching part to energy, in order to make the material of the attaching part mechanically firmer;
molding the attaching part to at least one given surface of the object.

15. The method according to claim 14, wherein the object is a medical instrument and/or an anatomical body structure and/or an implant.

16. A method for removing a marker attaching device according to claim 1 from an object, comprising at least one of the following steps:
subjecting the material of the attaching part to energy, in order to make the attaching part more plastically deformable;
spatially removing the attaching part from the object.

17. The method according to claim 16, wherein the object is a medical instrument and/or an anatomical body structure and/or an implant.

18. The method according to claim 11, wherein subjecting the material of the attaching part to energy includes subjecting it to mechanical oscillations and/or waves and/or thermal energy and/or electromagnetic radiation and/or a chemical reaction and/or electric current.

19. The use of a marker attaching device according to claim 1, wherein the attaching device is fastened to an object and/or is removed from an object.

20. The use according to claim 19, wherein the object is a medical instrument and/or an anatomical body structure and/or an implant.

21. The use according to claim 19, wherein the attaching device is fastened to the object in accordance with a method according to claim 11.

22. The use according to claim 19, wherein the attaching device is removed from the object in accordance with the method according to claim 16.

23. A computer program stored on a machine-readable medium for fastening a marker attaching device according to claim 1 to an object, comprising: code for subjecting the material of the attaching part to energy, in order to make the material of the attaching part more plastically deformable; and code for molding the attaching part to at least one given surface of the object.

24. The computer program according to claim 23, wherein the object is a medical instrument and/or an anatomical body structure and/or an implant.

25. The computer program according to claim 23, wherein it also includes code for making the attaching part firmer.

26. A computer program stored on a machine-readable medium for fastening a marker attaching device according to claim 1 to an object, comprising code for: subjecting the material of the attaching part to energy, in order to make the material of the attaching part mechanically firmer; and code for molding the attaching part to at least one given surface of the object.

27. The computer program according to claim 26, wherein the object is a medical instrument and/or an anatomical body structure and/or an implant.

28. A computer program stored on a machine-readable medium for removing a marker attaching device according to claim 1 from an object, comprising: code for subjecting the material of the attaching part to energy, in order to make the attaching part more plastically deformable; and code for spatially removing the attaching part from the object.

29. The computer program according to claim 28, wherein the object is a medical instrument and/or an anatomical body structure and/or an implant.

30. The computer program according to claim 23, wherein the code for subjecting the material of the attaching part to energy includes code for subjecting it to mechanical oscillations and/or waves and/or thermal energy and/or electromagnetic radiation and/or a chemical reaction and/or electric current.
